(19) United States
(12) Patent Application Publication
(10) Pub. No.: US 2003/0188744 A1
(43) Pub. Date: Oct. 9, 2003

(54) Automatic control system for rebreather

(76) Inventors: Alexander Roger Deas, Edinburgh (GB); Marat Vadimovich Evtukhov, St. Petersburg (RU)

Correspondence Address: Maria Nilova, Office 501, 58, Moika Embankment, St. Petersburg, 190000, RU

(21) Appl. No.: 10/425,653

(22) Filed: Apr. 30, 2003

Related U.S. Application Data
(63) Continuation-in-part of application No. PCT/RU01/00483, filed on Oct. 31, 2001.

(51) Int. Cl. A61M 16/00

(52) U.S. Cl. 128/201.27; 128/204.26

(57) ABSTRACT

Automatic control system for a rebreather, the automatic control system comprising sensors, a microcontroller and an indicator, the microcontroller being adapted to analyse readings of the sensors and, when abnormal readings are detected, actuate a bailout, generate a safety instruction to the diver and display this instruction on the indicator. The re-breathing apparatus has a bailout system automatically activated in an emergency, where the breathing circuit is shut off, and the diver starts inhaling directly from the breathable gas supply and exaling to the environment.

With the system of the invention, the diver is provided with information on the cause of this or that situation, together with clear instructions, so that the diver does not have to analyse the figures and take decision in stress situation.

### FIELD OF THE INVENTION

The present invention relates generally to diving systems and more particularly to automatic control system for a rebreather.

### BACKGROUND OF THE INVENTION

Self-contained underwater re-breathing apparatus or rebreathers are well known in the art. As the name implies, a rebreather allows a diver to “re-breathe” exhaled gas. Rebreathers consist of a breathing circuit from which the diver inhales and into which the diver exhales. The breathing circuit generally includes a mouthpiece in communication with an inlet to and outlet from, a scrubber canister for scrubbing CO₂ from the exhaled gas. At least one variable-volume container known as “counterlung” is incorporated in the breathing circuit. Exhaled gas fills the counterlung. Diver's inhalation draws the exhaled gas from the counterlung through the scrubber canister. CO₂-depleted gas from the scrubber canister is fed again to the mouthpiece and the diver's lungs.

A typical rebreather further includes an injection system for adding fresh breathable gas from at least one gas cylinder to the breathing circuit. It is vital to provide proper physical parameters (such as partial pressure of oxygen or PPO₂) of the breathing gas mixture inside the breathing circuit in accordance with pressure (determined by the depth of diving). This can be achieved by controlling said injection, which can be operated manually or automatically. In simple cases, that is small and constant depths, manual control can be employed, usually limited to adjusting a regulator for feeding breathable gas to a predetermined PPO₂. More or less complex diving profile at substantial depths requires automatic control.

Thus, up-to-date rebreathers usually have an automatic control system including a microcomputer provided with sensors for monitoring physical parameters in the breathing circuit and controlling the feeding of breathable gas to the breathing circuit in accordance with said physical parameters.

Usually, automatic control system for a rebreather such as described in U.S. Pat. No. 6,003,513 to Readey, et al. includes manual controls and an indicator typically located in a hanset. The indicator displays readings from the sensors. The diver can analyse the displayed readings and control some functions of the rebreather. In the case of an emergency, however, it does not always happen that a diver facing a dangerous situation under water keeps cool, makes a proper analysis and performs necessary actions.

Many conventional rebreathers are provided with bail out means to support the diver's life in case of system failure. An example is U.S. Pat. Nos. 4,964,404 and 5,127,398 by Stone. However, typically, bail out means shall be activated manually.

At the same time, a diver not always has sufficient time and knowledge to make an appropriate and adequate decision of switching the system into bail out mode. Often, it is a hard task for an average skilled diver to analyse the system parameters that are shown at the display in a short time period and make an optimal decision. The disadvantage of the manual bail-out is becoming apparent especially in cases where late or, contrary, early bail out can result in the exacerbation of the critical situation, or even, in a serious threat to a diver's life.

For example, if the oxygen supply is inadvertently switched off, contemporary CCRs will flag a problem by signalling alarms, and their display will show low ppO₂ levels. A diver may panic and ascend, but in doing so, ppO₂ will fall further, to the extent he may pass out or die before reaching the surface.

### BRIEF SUMMARY OF THE INVENTION

It is an object of the present invention is to provide an automatic control system for a rebreather with improved life-supporting characteristics..

A further object of the present invention is to provide an automatic control system for a rebreather with an automatic bailout system.

A further object of the present invention is to provide an automatic control system for a rebreather which helps the diver to take a right decision in an emergency.

These objects are achieved by providing an automatic control system for a rebreather, the automatic control system comprising sensors, a microcontroller and an indicator, the microcontroller being adapted to analyse readings of the sensors and, when abnormal readings are detected, actuate a bailout, generate a safety instruction to the diver and display this instruction on the indicator.

Thus, in addition to actual figures, the diver is provided with information on the cause of this or that situation, together with clear instructions, so that the diver does not have to analyse the figures and take decision in stress situation.

These objects can also be achieved by providing an automatic control system for a rebreather comprising a breathing circuit and a breathable gas supply in communication with the breathing circuit through a pressure differential control valve, the automatic control system comprising sensors, an indicator and a microcontroller adapted to analyse readings of the sensors and, when abnormal readings are detected, actuate a bailout, generate a safety instruction to the diver and display this instruction on the indicator, wherein the breathing circuit further includes a shut-off valve upstream the pressure differential control valve, and said bailout is activated by closing the shut-off valve.

Thus, bailout is activated automatically, and the diver does not have to take a decision in a stress situation.

The sensors can include an oxygen sensor, and bailout can be actuated when ppO₂ is low. Further, a carbon dioxide sensor can be among the sensors, and bailout can be actuated when pp CO₂ is high. A humidity sensor can also be used, and high humidity can trigger bailout.

Preferably, the indicator is located in a handset electrically connected to the microcontroller, and readings of the sensors are displayed on the indicator.

### BRIEF DESCRIPTION OF THE SEVERAL VIEWS OF THE DRAWING

These and other features, objects, and advantages of the present invention will be better appreciated from an understanding of the operative principles of a preferred embodiment as described hereinafter and as illustrated in the accompanying drawings wherein:

FIG. 1 is a schematic view of a rebreather according to the present invention;

FIG. 2 is a sectional view of a mouthpiece for a rebreather of the present invention;

FIG. 3 is a block diagram illustrating automatic control system for a rebreather according to the present invention; and

FIG. 4 is two sectional views of a mouthpiece for a rebreather of the present invention, wherein the mouthpiece is in open and closed state; and

FIG. 5 is a perspective view of a mouthpiece for a rebreather of the present invention.

### DETAILED DESCRIPTION OF THE INVENTION

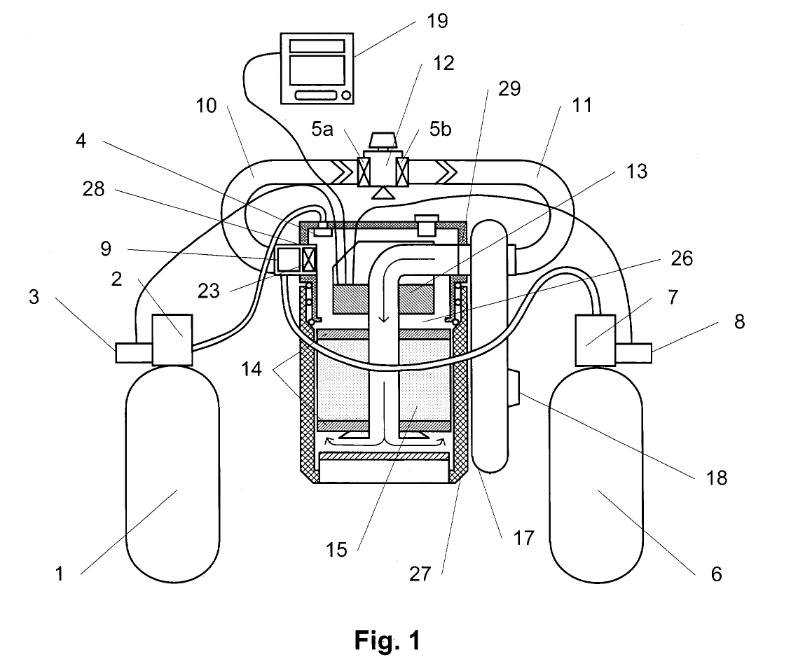

One embodiment of a self-contained underwater re-breathing apparatus according to the invention is shown schematically in FIG. 1, the rebreather including a breathing circuit defined by a mouthpiece 12 in communication with a scrubber canister 27. Exalation hose 11 provides fluid communication of an outlet of the mouthpiece 12 with a counterlung 17 which is in turn in communication with an inlet 29 of the scrubber canister 27. Counterlung 17 is a variable-volume container in the form of a bag for receiving exhaled gas. To throw off an exessive pressure from the breathing circuit a pressure-activated valve 18 is provided in the counterlung 17. Inhalation hose 10 provides fluid communication of an inlet of the mouthpiece 12 with an outlet 28 of the scrubber canister 27. To ensure that exhaled gas is fed to hose 11, and inhaled gas is fed from hose 10, check valves 5a and 5b are provided at the inlet and outlet, respectively, of the mouthpiece.

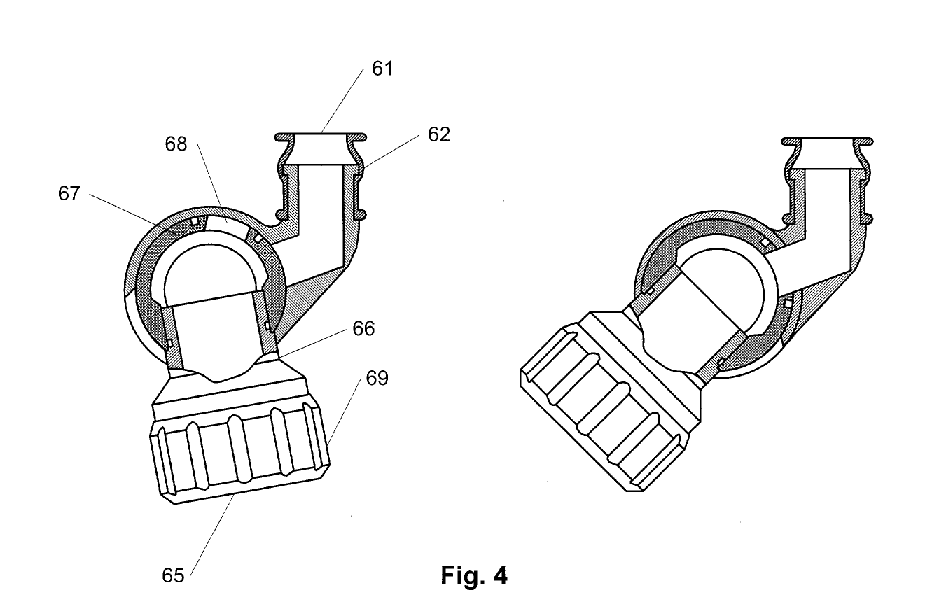

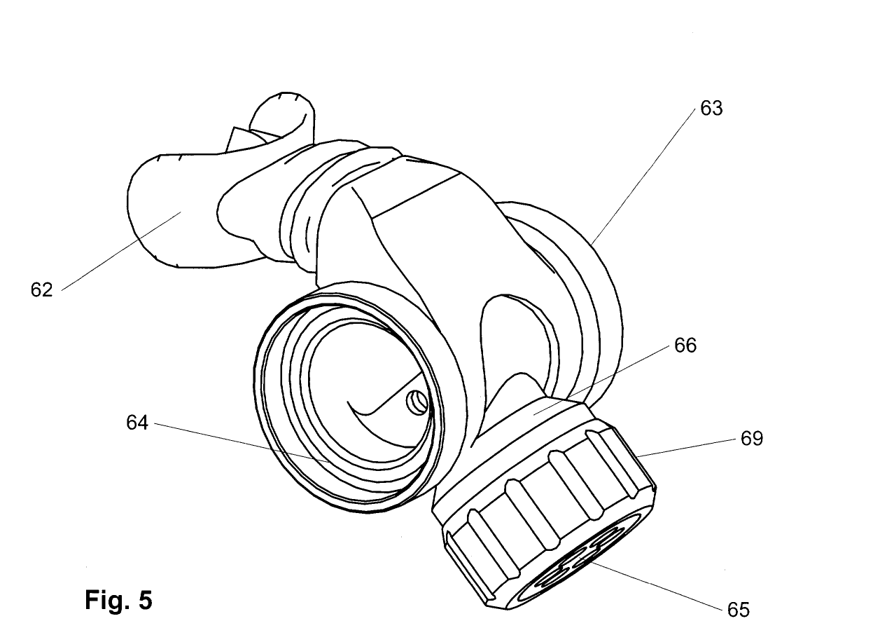

The mouthpiece 12 shown in FIGS. 4 and 5 is a hollow housing having a breathing opening 51 terminating in a rubber mouth bit piece 52, inlet 53 from and outlet 54 to, the breathing circuit, and an exhaust opening 55. The exhaust opening 55 is formed as a stub tube 56 having a pressure-activated exhaust valve. Detailed structure of the exhaust valve is neither disclosed herein nor presented in the drawings because it is well known in the art and widely used in open-circuit SCUBAs. The exhaust valve can open to the environment at a predetermined pressure which can be adjusted manually by rotating a knob 59. Normally, the exhaust valve is adjusted to a pressure higher than normal pressures in the breathing circuit, but not above the highest pressure that can be created by the diver's lungs.

A means for shutting off the breathing opening 51 are provided in the mouthpiece 12. A part of the mouthpiece housing between the inlet 53 and the outlet 54 is cylindrical, and has a cylindrical routable insert 57 therein, the insert being fixed to the stub tube 56. By rotating the insert, its opening 58 can either be aligned or misaligned with the breathing opening 51. The insert 57 is rotated manually by acting on the stub tube 56. A diver can need to shut off the breathing opening 51 in some emergency situations where he has to take the mouthpiece out of his mouth, e.g. to start breathing from a backup breathing circuit (not disclosed herein).

Referring back to FIG. 1, the scrubber canister 27 (adapted to be secured on the diver's back) comprises a scrubber unit 15 usually in the form of a sheet roll sandwiched between filters 14. Alternatively, scrubber unit 15 can be a granular filling. Scrubber unit 15 contains chemicals capable of absorbing CO₂ from exhaled gas passed there through. In the scrubber canister 27 downstream the scrubber unit 15 a chamber 26 is formed, partly occupied by an automatic control system 13 described below. Thus, electronics of the automatic control system is located within a secure, moisture-proof housing of the canister.

The gas flow in the scrubber canister 27 is arranged in such a way that exhaled gas entering the inlet 29 passes through the scrubber unit 15 to the chamber 26 and out to the outlet 28.

An injection system for adding fresh breathable gas to the breathing circuit includes an oxygen cylinder 1 containing compressed oxygen and communicated to the breathing circuit, namely, to chamber 26 via solenoid control valve 4. The cylinder has a pressure regulator 2 for adjusting pressure of oxygen injected to the breathing circuit. The injection system further includes diluent gas cylinder 6 containing compressed diluent gas, which is usually a standard breathable mixture of oxygen and a nontoxic inert gas. Cylinder 6 has pressure regulator 2 for adjusting pressure of diluent gas injected to the breathing circuit. This cylinder is in fluid communication with chamber 26 via pressure-activated regulator 9 having a second stage control valve.

The automatic control system 13 includes a microcomputer electrically connected with sensors for monitoring physical parameters both outside and inside the breathing circuit. On the other hand, the microcomputer is electrically connected with the solenoid of oxygen valve 4 for controlling the injection of oxygen into the breathing circuit in accordance with current values of the physical parameters monitored by the sensors. Further, the microcomputer is electrically connected with a handset 19 having an indicator and manual controls.

The microcomputer includes a microcontroller 55 responsible for adding oxygen to the breathing circuit and a microcontroller 56 for providing information on diving profile to the handset.

Among the sensors are oxygen sensors 41, a carbon dioxide sensor 42, an inert gas sensor 43, temperature sensors 44, and a water sensor 46. These sensors are electrically connected to the microcomputer. The sensors, especially carbon dioxide sensor 2, are disposed in the vicinity of oxygen supply valve 4, so that dry oxygen is blown across the sensors. This avoids humidity condensation and provides higher accuracy.

For monitoring the amount of oxygen and diluent gas in cylinders 1 and 6 these cylinders are provided with respective sensors 3 and 8 electrically connected to the microcomputer. Readings from these sensors are displayed by the handset.

A solenoid shut-off valve 23 is incorporated in the breathing circuit upstream the control valve. Preferably, shut-off valve 23 is disposed within the canister 27. In this embodiment, shut-off valve 23 is disposed in the scrubber outlet 28. Solenoid of shut-off valve 23 is electrically connected to the microcomputer. Thus, the solenoid is safely and conveniently disposed within the canister 27 in the vicinity of other electronics.

During the dive, the diver exhales to the breathing circuit. Through check valve 5b exhaled gas enters hose 11 and fills counterlung 17. Check valve 5a prevents the exhaled gas from entering hose 10. When the diver inhales, his lungs create a vacuum which draws the exhaled gas from counterlung 17 to scrubber canister 27 and further downstream the breathing circuit. In the scrubber canister, the exhaled gas is scrubbed from CO₂ to maintain partial pressure of carbon dioxide or PPCO₂ downstream the scrubber less than 0.005 ATA.

CO₂-depleted gas is fed to hose 10 and, through check valve 5a, back to mouthpiece 12, and the diver's lungs, while check valve 5b prevents gas in hose 11 from entering the mouthpiece. PPO₂ in the exhaled gas is decreased due to metabolism. When O₂ sensors detect a decreased PPO₂ in the breathing circuit as compared to a predetermined level, microcomputer activates solenoid control valve 4 to add deficient oxygen to the breathing circuit.

When the diver descends, the outside pressure increases. This leads to pressure difference between the breathing circuit and the outside. Under this pressure difference, regulator 9 is activated providing a corresponding rise of pressure in the breathing circuit by adding some diluent gas from cylinder 6.

Abnormal readings of at least one sensor are analysed by the automatic control means. If hazard to the diver's life is detected, shut-off valve 23 is closed.

This will close the breathing circuit, and an open-circuit bailout will automatically be actuated. More specifically, vacuum created by the diver's inhalation will cause pressure difference between the breathing circuit and the outside. This will open pressure-activated regulator 9, and diluent gas will come from cylinder 6 to the part of the breathing circuit downstream shut-off valve 23, that is, to hose 10 and inlet 5a to mouthpiece 12. Thus, the diver will inhale diluent gas from cylinder 6.

When the diver exhales, the pressure downstream the mouthpiece outlet opening will increase because the breathing circuit is shut off. The increased pressure will open the exhaust valve, and the exhaled gas will be released to the environment. To facilitate exhalation, the diver can adjust the exhaust valve to a lower pressure. However, even if he does not do that, the exhaled gas will still be exhausted because, as mentioned above, the exhaust valve is normally adjusted to a pressure not higher than the highest pressure that can be created by the diver's lungs.

This means that the diver can breathe in an open-circuit mode. More specifically, the diver inhales from cylinder 6 through pressure-activated regulator 9, hose 10, and mouthpiece 12, and exhales through the exhaust valve. Thus, a part of the existing closed circuit is used for bailout, and no separate bailout circuit is provided. Therefore, there is no need to incorporate in the mouthpiece means for switching from one breathing circuit to another, and the mouthpiece can be kept smaller and simpler. As described above, switching to bailout is fully automated, so that no actions are required from the diver.

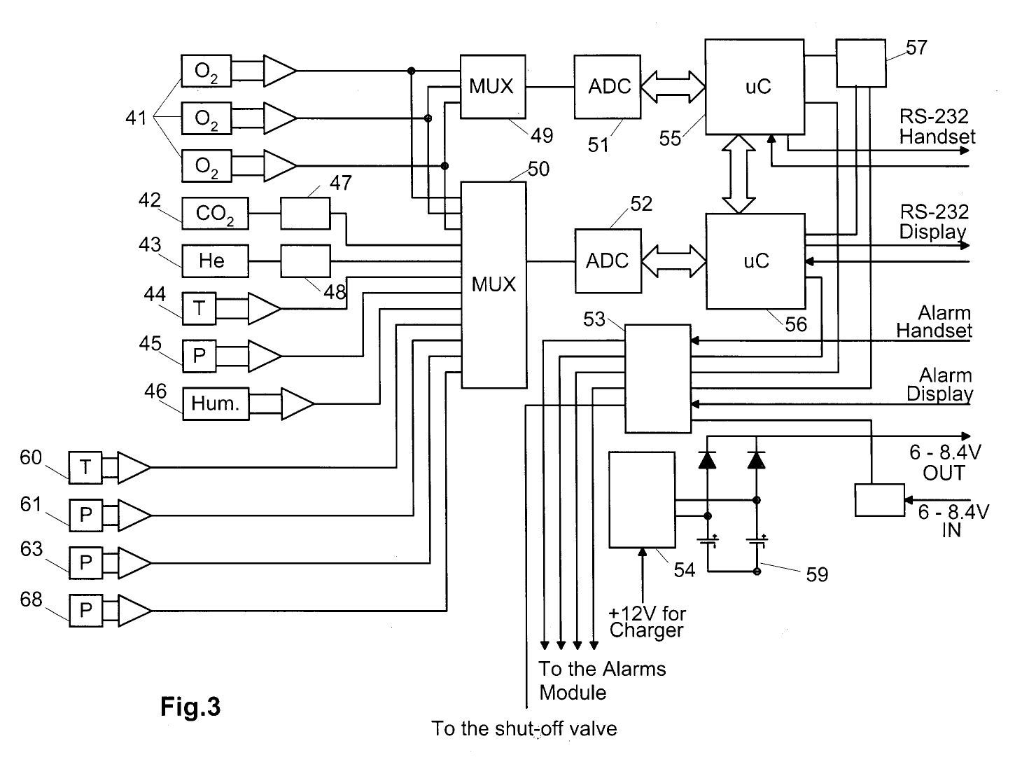

Automatic control system 13 is described below in more details with reference to a circuit diagram shown in FIG. 3.

The automatic control system 13 maintains the required level of ppO₂ in the breathing circuit, monitors gas mixture, and provides the diver with life critical information on the diving process.

Output signals from oxygen sensors 41 are transmitted through three-to-one analogue multiplexer 49 to the input of the analogue-to-digital converter 51. Oxygen control microcontroller 55 regularly reads data from analogue-to-digital converter 51 and calculates the partial pressure of oxygen in the breathing circuit. Microcontroller 55 takes the median of the two closest signals as already mentioned above as being the true oxygen value. The result is used to maintain an accurate ppO₂ in the breathing circuit, within ppO₂ of +/−0.05. The sensors are located adjacent to the output 28 of chamber 26.

When the level of the ppO₂ in the breathing gas is below a predefined level, microcontroller 55 generates signals to solenoid valve circuitry 57 to activate oxygen valve 4 to feed a portion of oxygen from cylinder 1 to the breathing circuit. In case of failure, solenoid valve circuitry 57 produces an alarm signal and sends it to alarm circuitry 53 and further to shut-off valve 23 in order to activate the bailout system. Other situations in which the bailout system is activated are indicated in Table 1 below.

From the alarm circuitry 53, the alarm signal also comes to an alarms module (not shown). The alarms module has a buzzer and ultrabight red LED. This module is fully controlled by the alarm circuitry 53. Alarms module is usually located on the diver's mask in such a way that the diver can see the LED and hear the buzzer.

To provide the diver with information on the current state of the diving process, automatic control system 13 includes breathing gas monitor microcontroller 56. Signals from sensors 41, 44-46, carbon dioxide monitor 47, helium monitor 48, ambient water temperature sensor 60, ambient pressure sensors 61, and pressure sensors 3, 8 are transmitted through multiplexer 50 to the input of analog-to digital converter 52. The microcontroller 56 reads data from analog-to digital converter 52, computes the current content of the breathing gas mixture, and transmits the information to display module 19. In case of abnormal readings of one or more sensors, the content of the breathing gas will be found abnormal. This will lead to activation of the alarm module and bailout system. Specific situations in which the bailout system is activated are indicated in Table 1 below.

The automatic control system 13 is powered from battery pack 59. When the batteries are discharged, the diver has an opportunity to re-charge the batteries. Automatic control system 13 has a charge unit 54 with two independent charge channels. A voltage of +12V is used for charging.

The estimated service life of the scrubber is calculated based on his design life each time a new scrubber is fitted. Before diving, the system requests from the user the intended duration of his dive. If this duration exceeds the estimated scrubber life, the system rejects the dive and warns “No dive”, “Insufficient scrubber”.

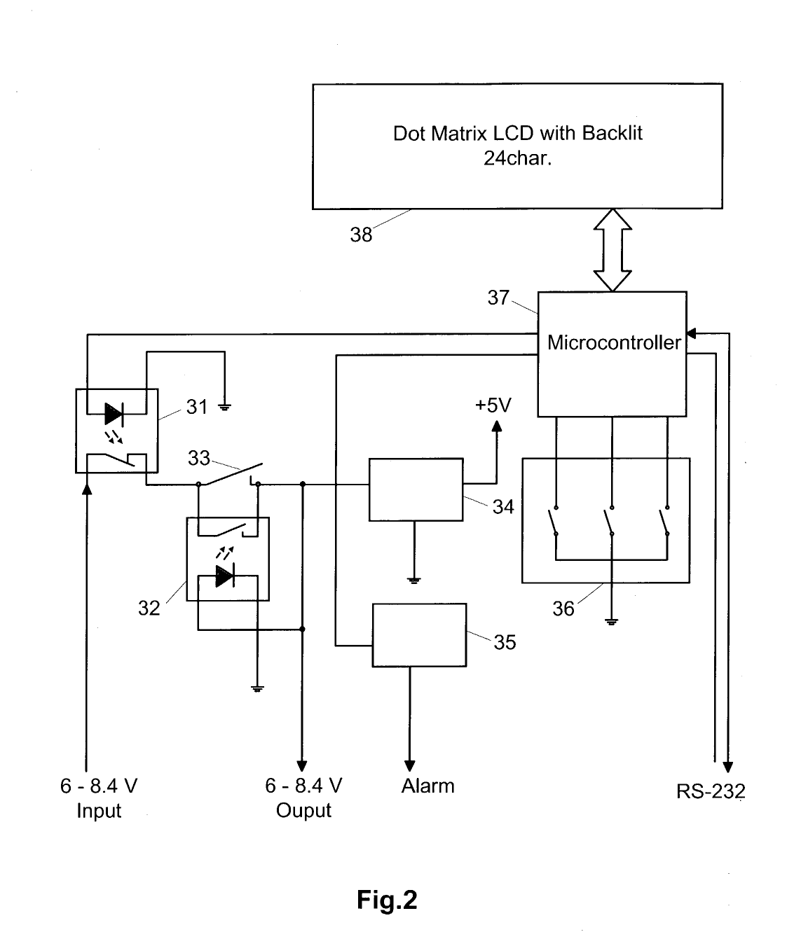

FIG. 2 is a circuit diagram representing handset 19 in accordance with the preferred embodiment of the present invention.

According to the present embodiment, handset 19 allows the diver to set the desired parameters of the dive, check manually gas control electronics, and calibrate the oxygen sensors.

The diver switches on power by initiating the normally opened reed switch 33. The power from the batteries, coming across a normally closed solid-state relay 31 and the closed contact of reed switch 33, activates a normally opened solid-state relay 32. The contact of the relay 32 will be closed, thus powering the handset and electronics. To switch power off electronics of the rebreather, at least two of reed Hall-effect switches 36 should be pressed, then, after the confirmation by the diver, the power will be switched off by opening the closed contact on relay 31. This prevents accidental switching the power off during the dive.

The handset has its own alarm circuitry. Alarm signal is generated in case of microcontroller 37 or power failure.

The handset is powered from the 5V power regulator 34 with a low dropout.

Initiating Hall-effect switches 36 defines a change in different modes of operation of the rebreather. Microcontroller 37 decodes the combination of the switches and passes messages to the diver on a dot matrix LCD 38 with a red 680 nm backlit. Each change of state of the Hall-effect switches 36 activates the backlit diode of the LCD for several seconds, and the diver will hear a short sound from the buzzer. Thus, the diver is provided with a means for controlling the adequacy of instructions. The handset communicates with the automatic control system 13 via RS-232 interface. Handset shows all key data and operating instructions in the LCD 38, which is switched on in the event of alarm, and/or when any button is pressed.

The LCD 38 displays:

DIVE DATA: Total dive time (h, mm), Max Depth (ddd), Time to surface (h, mm), Ceiling (nnn), Time at ceiling (h, mm, ss), Gas %: He, N₂, O₂, Water Temperature, Ascent rate (+/−ft/s or m/s);

INSTRUCTION DISPLAY: 24 char alpha numeric, red backlit;

CAUSE DISPLAY: 24 char alpha numeric, red backlit;

CRITICAL DATA: ppN₂, ppO₂, ppCO₂, Battery (%);

SENSORS: Select O₂ (x3), He, ppCO₂, Battery V, Idd, Humidity;

GAS SUPPLIES: O₂ cylinder pressure, Diluent gas cylinder pressure, Scrubber life.

An important feature of the invention is that in addition to actual figures, the diver is provided with information on the cause of this or that situation, together with clear instructions, so that the diver does not have to analyse the figures and take decision in stress situation.

An approximate list of potentially dangerous situations in which instructions to the diver are generated is shown in Table 1. Situations 1, 3, 4, 6, and 7 can be managed, and bailout is not necessary. Therefore, the shut-off valve remains open, whereas the diver is instructed on further actions. In situations 2, 5 and 8-11 the diver faces a deadly danger, therefore the shut-off valve is closed and bailout is activated.

TABLE 1

| NO. | TRIGGER | INSTRUCTION | CAUSE | BUZZER | LED | SHUT-OFF VALVE |
|---|---|---|---|---|---|---|
| 1 | ppO₂ < set ppO₂-0.3 | “Inject O₂”/“Do NOT ascend” | “ppO₂ is low” | On slow | On slow | Open |
| 2 | ppO₂ < 0.20 | “Bail out NOW!”/ | “No Oxygen” | On fast | On fast | Closed |
| | | “Do NOT ascend on RB” | | | | |
| 3 | On standby battery | “Abort Dive” | “On standby power” | Int | Int | Open |
| 4 | ppCO₂ > 0.05 | “Abort Dive” | “High ppCO₂” | Int | Int | Open |
| 5 | ppCO₂ > 3.5 | “Bail out NOW!” | “Scrubber failure” | On fast | On fast | Closed |
| 6 | ppN₂ > 4 | “Ascend slowly” | “N₂ Narcosis” | Int | Int | Open |
| 7 | ppO₂ > 1.6 | “Flush & Shut off O₂” | “O₂ solenoid stuck on” | On med | On med | Open |
| 8 | Depth < 1 m and checks not complete | “No dive” | “Checks not complete” | Off | off | Closed |
| 9 | Current > 60 mA av. 10 sec | “Bail out NOW” | “System failed (Icc H)” | On fast | On fast | Closed |
| 10 | Current < 10 mA av. 10 sec | “Bail out NOW” | “System failed (Icc L)” | On fast | On fast | Closed |
| 11 | Humidity sensor RH > 98% | “Bail out NOW” | “System is Flooding” | On fast | On fast | Closed |

We claim:

1. Automatic control system for a rebreather, the automatic control system comprising sensors, a microcontroller and an indicator,
the microcontroller being adapted to analyse readings of the sensors and, when abnormal readings are detected, actuate a bailout, generate a safety instruction to the diver and display this instruction on the indicator.

2. Automatic control system according to claim 1, wherein the sensors include a oxygen sensor, and bailout is actuated when ppO₂ is low.

3. Automatic control system according to claim 1, wherein the sensors include a carbon dioxide sensor, and bailout is actuated when pp CO₂ is high.

4. Automatic control system according to claim 1, wherein the sensors include a humidity sensor, and bailout is actuated when humidity is high.

5. Automatic control system according to claim 1, wherein the indicator is located in a handset electrically connected to the microcontroller.

6. Automatic control system according to claim 1, wherein readings of the sensors are displayed on the indicator.

7. Automatic control system according to claim 1, wherein, when abnormal readings are detected, the microcomputer further generates a report on the cause of the situation and displays this report on the indicator.

8. Automatic control system for a rebreather comprising a breathing circuit and a breathable gas supply in communication with the breathing circuit through a pressure differential control valve, the automatic control system comprising sensors, an indicator and a microcontroller adapted to analyse readings of the sensors and, when abnormal readings are detected, actuate a bailout, generate a safety instruction to the diver and display this instruction on the indicator, wherein the breathing circuit further includes a shut-off valve upstream the pressure differential control valve, and said bailout is activated by closing the shut-off valve.

9. Automatic control system according to claim 8, wherein the sensors include a oxygen sensor, and bailout is actuated when ppO₂ is low.

10. Automatic control system according to claim 8, wherein the sensors include a carbon dioxide sensor, and bailout is actuated when ppCO₂ is high.

11. Automatic control system according to claim 8, wherein the sensors include a humidity sensor, and bailout is actuated when humidity is high.

12. Automatic control system according to claim 8, wherein the indicator is located in a handset electrically connected to the microcontroller.

13. Automatic control system according to claim 8, wherein readings of the sensors are displayed on the indicator.

14. Automatic control system according to claim 8, wherein, when abnormal readings are detected, the microcomputer further generates a report on the cause of the situation and displays this report on the indicator.
